SEQUENCE LISTING (2 sequences)

SEQ ID NO: 1 - protein, 772 aa, Arabidopsis thaliana
MGTKFLALGLSLCLVLSSFYQVSCQDEGTGSLSTLDLIEHEYQTSVNSLQGNEAVDQTETSGQKNSTVSDNNTISLSLSEEPALETLKESVDTSAELGAVTDEVDKPSSMLDHIELEFEAHINELKEAGSDGINKVEESKDDEEAARRHKMLEAIEREFEAAHAGFEQLKTDDSAQGLDDEQSAKRQSMLDEIERDFEAATKGLEQLKADDLTGINDEEHAAKRQKMLEEIEREFEEATKGLEELRHSTSSTDDEAQSAKRQNMLDEIEREFEAATSGLKELKINAHTVKDDVDDKEQDAKRQSMLDAIEREFEAVTESFKQLEDIADNKAEGDDESAKRQSMLDEIEREFEAATNSLKQLNLDDFSEGDDSAESARRNSMLEAIEREFEAATKGLEELKANDSTGDKDDDEHVARRKIMLEAIEREFEAATKGLEELKNESEQAENKRNSMLEAFEREFEAATNAKANGENSAKNPSTISTTVQKSSGGYNAGLEGLLKPADGVCGCFNKDKDGLQADTDSSINIAEILAEESKLQGSGTSRLTTSLNNLVDTHRKETSSKVGSVLGSSSSVTSTTSESAATSESIESLKQTLRKLRGLSARDLVNHPNFDAIIAAGTRYEVLSSASIGYISLLAKYKTVIKEGLEASQRVQIAQTRAKLLKETAMEKQRTVDSVFAAAKTTAQRGDALHIRIVAIKKLLAKLEAEKVDVDSKFTSLTTSLSELLKEASQAYEEYHEAVHKAKDEQAAEEFAVETTKRAEHIWVEFLSSLN

SEQ ID NO: 2 - protein, 524 aa, Arabidopsis thaliana
MVLQKLPLIGLLLLLTIVASPANADGPVCPPSNKLSRASFPEGFLFGTATAAYQVEGAINETCRGPALWDIYCRRYPERCNNDNGDVAVDFFHRYKEDIQLMKNLNTDAFRMSIAWPRIFPHGRKEKGVSQAGVQFYHDLIDELIKNGITPFVTVFHWDTPQDLEDEYGGFLSERIVKDFREYADFVFQEYGGKVKHWITFNEPWVFSHAGYDVGKKAPGRCSSYVNAKCQDGRSGYEAYLVTHNLLISHAEAVEAYRKCEKCKGGKIGIAHSPAWFEAHDLADSQDGASIDRALDFILGWHLDTTTFGDYPQIMKDIVGHRLPKFTTEQKAKLKASTDFVGLNYYTSVFSNHLEKPDPSKPRWMQDSLITWESKNAQNYAIGSKPLTAALNVYSRGFRSLLKYIKDKYANPEIMIMENGYGEELGASDSVAVGTADHNRKYYLQRHLLSMQEAVCIDKVNVTGYFVWSLLDNFEWQDGYKNRFGLYYVDFKNNLTRYEKESGKYYKDFLSQGVRPSALKKDEL

(19) United States
(12) Patent Application Publication
(10) Pub. No.: US 2014/0137289 A1
(43) Pub. Date: May 15, 2014

(54) Method For Accumulating A Protein In Plant Cell

(71) Applicants: Yoichi Ogawa, Wako-shi (JP); Yasuhiro Kondo, Wako-shi (JP); Ikuko Nishimura, Kyoto (JP); Tomoo Shimada, Kyoto (JP); Makoto Shirakawa, Kyoto (JP)

(72) Inventors: Yoichi Ogawa, Wako-shi (JP); Yasuhiro Kondo, Wako-shi (JP); Ikuko Nishimura, Kyoto (JP); Tomoo Shimada, Kyoto (JP); Makoto Shirakawa, Kyoto (JP)

(73) Assignee: Kyoto University, Kyoto-shi (JP)

(21) Appl. No.: 14/000,583

(22) PCT Filed: Feb. 21, 2012
(86) PCT No.: PCT/JP2012/054122

§ 371 Date: Oct. 9, 2013

(30) Foreign Application Priority Data
Feb. 22, 2011 (JP) 2011-035558

(51) Int. Cl. C12P 21/02

(52) U.S. Cl. CPC C12P 21/02
USPC 800/278; 435/69.8; 800/298; 800/320; 800/320.2

(57) ABSTRACT

A method for accumulating a protein in a plant cell, in which by co-expressing a gene which encodes a polypeptide having an ability to form an ER body, and a gene which encodes a target protein having an endomembrane system migration signal peptide at the N-terminus and having an ER retention signal peptide at the C-terminus in the plant cell, the target protein or a protein in which the N-terminus domain of the target protein is lacking is accumulated in an ER body formed in the plant cell.

### TECHNICAL FIELD

The present invention relates to a method for accumulating a protein in a specific subcellular organelle of a plant cell and a transformed plant prepared by this method.

Priority is claimed on Japanese Patent Application No. 2011-035558, filed Feb. 22, 2011, the content of which is incorporated herein by reference.

### BACKGROUND ART

By the advancement in gene recombination techniques, it is now possible to introduce a gene which encodes a desired protein in a cultured cell and a cell of an individual plant, and to express the protein in a cell. In general, when a protein is expressed after a gene is introduced into a nuclear genome of a plant, in a case where only a gene region which encodes a desired protein is introduced into a cell, the expressed protein is accumulated in a cytoplasm. In addition, by introducing a gene which encodes a protein with several added amino acids which is called a signal peptide at the N-terminus of the desired protein, it is possible for the expressed protein to migrate to and accumulate in a place which is called a subcellular organelle such as an ER (endoplasmic reticulum), a vacuole and a chloroplast or an extracellular region (apoplast) (refer to Non-Patent Literature 1).

Further, there is also a technique used for performing from gene expression to protein accumulation in chloroplasts by introducing a gene which encodes a desired protein into the chloroplast genome (refer to Non-Patent Literature 2).

Also, as a subcellular organelle which accumulates a protein specifically, a protein body, an ER body and the like are known (refer to Non-Patent Literature 3). The protein body is localized in only a seed of a plant, and the ER body is localized in only a seedling of a Capparales plant including Cruciferae and the like and in Arabidopsis thaliana which is included in Capparales Cruciferae, function failure mutation occurs in an nai2 gene in an ER body formation mutant, and by introducing a wild type of nai2 gene into the mutant, an ER body is formed, and Pyk10 which is a β-glucosidase is accumulated in the ER body; however, in an ER body formation mutant, Pyk10 is localized distributively in the entire ER and the like have been reported (refer to Non-Patent Literatures 4 to 8).

On the other hand, in the related art in which a protein is accumulated in an existing subcellular organelle, there is a case where an adverse effect occurs in the accumulating subcellular organelle, the function of the cells thereof, and the growth of plants, according to the protein type. For example, a plant which expresses and accumulates a diastatic enzyme which hydrolyses a plant cell wall-derived cellulose to sucrose is expected to be a material suitable for bio-ethanol production. However, it has been reported that in the rice in which such a diastatic enzyme is expressed and accumulated artificially, various physiological and morphological abnormalities occur (refer to Non-Patent Literature 9).

### CITATION LIST

### Non-Patent Literature

[NPL 1] Twyman, et al., Trends Biotechnol. 2003, 21(12):570-578.
[NPL 2] Verma, et al., Plant Physiol. 2007, 145(4):1129-1143.
[NPL 3] Hara-Nishimura I, et al., Plant Physiol. 2004, 136(3):3435-3439.
[NPL 4] Yamada, et al., Plant Cell. 2008, 20(9):2529-2540.
[NPL 5] Yamada, et al., Plant Signal Behay. 2009, 4(9):849-852.
[NPL 6] Matsushima R, et al., Plant J. 2003, 33(3):493-502.
[NPL 7] Matsushima R, et al., Plant Physiol. 2002, 130(4):1807-1814.
[NPL 8] Matsushima R, et al., Plant Cell. 2004, 16(6):1536-1549.
[NPL 9] Nigorikawa M, et al., “Abs # P10033: Effects of overexpression of a cellulase gene on rice development”, [on line], 2009, Plant Biology, [searched on Dec. 6, 2010], internet<URL: http://abstracts.aspb.org/pb2009/public/P10/P10033.html>
[NPL 10] Cornelissen, et al., Nucleic Acids Res. 1987, 15(17):6799-6811.
[NPL 11] Chen, et al., Plant Mol Biol. 2006, 62(6):927-936.
[NPL 12] Nishimura et al., Nature Protocols 2006, 1:2796-2802.
[NPL 13] Kawazu et al., 1999, Journal of bioscience and bioengineering, 88:421-425.
[NPL 14] Kimura et al., Applied microbiology and biotechnology, 2003, 62:374-379.
[NPL 15] Ziegler, et al., Mol Breed. 2000, 6:37-46.
[NPL 16] Kengen, et al., Eur J. Biochem. 1993, 213:305-312.

### SUMMARY OF INVENTION

### Technical Problem

In order to stably accumulate a large amount of a desired protein in a plant, it is important that the obstacles such as growth abnormality do not occur in a plant by the protein accumulation. For example, it may be expected that by accumulating an overexpressed protein in a protein body or an ER body, the effect on the plant itself by the protein is sufficiently decreased while the protein of high concentration is accumulated in a plant. However, since plant species, time, and organs in which a protein body and an ER body are formed are limited, a protein body and an ER body are not suitable as an accumulation organ for the mass production of a protein.

The present invention is to provide a method which stably accumulates a desired protein in a plant cell and a plant, and a transformed plant in which a protein is accumulated.

### Solution to Problem

The present inventors, as a result of intensive studies to solve the above problems, found that by introducing an nai2 gene into a plant other than a Capparales Cruciferae plant, an ER body can be formed and in a plant in which an ER body is newly formed, by expressing a protein with an added endomembrane system migration signal peptide, and an ER retention signal peptide, the protein is accumulated in the ER body, and completed the present invention.

That is, the present invention provides the followings.

(1) A method for accumulating a protein in a plant cell, in which by co-expressing a gene which encodes a protein having an ability to form an ER body, and a gene which encodes a target protein having an endomembrane system migration signal peptide at the N-terminus and having an ER retention signal peptide at the C-terminus in a plant cell, the target protein or a protein in which the N-terminus domain of the target protein is lacking is accumulated in an ER body formed in the plant cell.

(2) The method for accumulating a protein in plant cell according to (1) in which a protein having the ability to form an ER body is a polypeptide selected from any one of (a) to (d) below;

(a) A polypeptide formed of an amino acid sequence represented by seq id no. 1,

(b) A polypeptide formed of an amino acid sequence in which one or several amino acids are lacking, substituted or added, in the amino acid sequence represented by seq id no. 1, and having an ability to form an ER body,

(c) A polypeptide having homology of 80% or more with the amino acid sequence represented by seq id no. 1 and having an ability to form an ER body,

(d) A polypeptide having homology of 80% or more with an amino acid sequence formed of the 473rd to the 772nd amino acid in the amino acid sequence represented by seq id no. 1 and having an ability to form an ER body.

(3) The method for accumulating a protein in plant cell according to (1) or (2), in which the target protein is a protein fused with another polypeptide at the C-terminus of Pyk10 protein.

(4) The method for accumulating a protein in plant cell according to any one of (1) to (3), in which the plant cell is a cell in plant individual.

(5) A transformed plant, into which an expression vector having a base sequence which encodes a protein having an ability to form an ER body and an expression vector having a base sequence which encodes a target protein are introduced, or an expression vector having both a base sequence which encodes a protein having an ability to form an ER body and a base sequence which encodes a target protein is introduced,

in which the target protein has an endomembrane system migration signal peptide at the N-terminus, and has an ER retention signal peptide at the C-terminus, and

in at least one cell in the plant, a target protein or a protein in which the N-terminus region of the target protein is lacking is accumulated in an ER body formed in the cell.

(6) The transformed plant according to (5), in which a protein having the ability to form an ER body is a polypeptide selected from any one of (a) to (d) below;

(a) a polypeptide formed of amino acid sequence represented seq id no. 1,

(b) a polypeptide formed of an amino acid sequence in which one or several amino acids are lacking, substituted or added in the amino acid sequence represented by seq id no. 1, and having the ability to form an ER body,

(c) a polypeptide having homology of 80% or more with the amino acid sequence represented by seq id no. 1 and having the ability to form an ER body, and

(d) a polypeptide having homology of 80% or more with the amino acid sequence formed of the 473rd to the 772nd amino acid in the amino acid sequence represented by seq id no. 1, and having the ability to form an ER body.

(7) The transformed plant according to (5) or (6) in which the target protein is a protein fused with another polypeptide at the C-terminus of Pyk10 protein.

(8) The transformed plant according to any one of (5) to (7) which is a monocot.

(9) The transformed plant according to any one of (5) to (7) which is a liliaceae plant or a gramineous plant.

(10) The transformed plant according to any one of (5) to (7) which is a rice plant.

(11) A plant which is a next generation individual or a cloned individual of the transformed plant according to any one of (5) to (10).

### Advantageous Effects of Invention

By a method for accumulating a protein into a plant cell of the present invention, the effect on a plant cell and a plant individual resulting from an overexpression can be sufficiently decreased while a desired protein can be accumulated in the plant cell.

In addition, a transformed plant of the present invention can comparatively stably accumulate a foreign protein in an ER body.

### BRIEF DESCRIPTION OF DRAWINGS

FIG. 1 is a diagram schematically showing each expression cassette prepared in Example 1 and Example 2.

FIG. 2 is an observed image of a cell into which each expression vector is introduced, by using a fluorescence microscope, in Example 1.

FIG. 3 is an observed image of a cell into which each expression vector is introduced, by using a fluorescence microscope, in Example 2.

### DESCRIPTION OF EMBODIMENTS

Hereinafter, the preferable examples of the invention will be described, but the invention is not limited to the examples. Within the scope of the purport of the invention, additions, omissions and substitutions of the constituents and other modifications are available.

A method for accumulating a protein in plant cells of the present invention is characterized in that a desired protein expressed by a transformation is accumulated in a subcellular organelle newly formed by a transformation. Specifically, by introducing a gene which encodes a protein having an ability to form an ER body in a plant cell, an ER body is formed in the plant cell, and then, a desired protein is accumulated in the ER body. A technique for preparing a new subcellular organelle which is specialized in such a protein accumulation has not yet been reported.

That is, a method for accumulating a protein in a plant cell of the present invention is a method for accumulating a protein in a plant cell, wherein by co-expressing a gene which encodes a protein having an ability to form an ER body (ER body formation related gene), and a gene which encodes a target protein having an endomembrane system migration signal peptide at the N-terminus and having an ER retention signal peptide at the C-terminus in a plant cell, the target protein or a protein in which the N-terminus region of the target protein is lacking is accumulated in an ER body formed in the plant cell. By expressing a gene which encodes a protein having the ability to form an ER body, an ER body is newly formed in a cell. In addition, when a gene which encodes a target protein having an endomembrane system migration signal peptide at the N-terminus, and the ER retention signal peptide at the C-terminus is expressed in the plant cell in which an ER body is formed, the expressed protein can be accumulated in the ER body.

In the present invention and the specification, a gene is a nucleic acid or derivative thereof which includes a base sequence in which a protein is encoded, and in which an encoded protein is synthesized by a transcription and translation mechanism of a cell, by being introduced into the cell. The gene includes not only a natural gene of an organism has but also a gene designed and synthesized artificially by gene recombination techniques.

Examples of a protein having an ability to form an ER body include an nai2 (seq id no. 1) of Arabidopsis thaliana, and TSK-associating protein1 (TSAI)/At1g52410, a protein which is encoded in At3g15960, homologue proteins of these proteins and the like. In order from the N-terminus, an nai2 has an endomembrane system migration signal peptide (the region of 1st to 24th amino acid in seq id no. 1), ten EFE repeats (the region of 98th to 472nd amino acid), and an nai2 domain (the region of 473rd to 772nd amino acid). TSAI, as nai2, has an endomembrane system migration signal peptide, ten EFE repeat, and nai2 domain and has a homology of 80% in an amino acid sequence. Forming an ER body in a plant other than Capparales including Cruciferae and the like by expressing nai2, a homologue protein thereof, and the like, was discovered by the present inventors.

Protein having an ability to form an ER body used in a method for accumulating a protein in a plant cell of the present invention is preferably a polypeptide selected from any one of (a) to (d) below.

(a) A polypeptide formed of an amino acid sequence represented by seq id no. 1.

(b) A polypeptide formed of an amino acid sequence in which one or several amino acids are lacking, substituted or added in the amino acid sequence represented by seq id no. 1, and having an ability to form an ER body.

(c) A polypeptide having homology of 80% or more with the amino acid sequence represented by seq id no. 1, and more preferably homology of 90% or more, and having an ability to form an ER body.

A homology of an amino acid sequence with the amino acid sequence represented by seq id no. 1 can be obtained using known programs such as Blast and the like.

(d) A polypeptide having a homology of 80% or more with an amino acid sequence formed of the 473rd to the 772nd amino acid in the amino acid sequence represented by seq id no. 1, and more preferably a homology of 90% or more, and having an ability to form an ER body.

A protein (polypeptide) having an ability to form an ER body refers to a protein in which an ER body is formed by expressing the protein in a plant cell. Whether a certain polypeptide has an ability to form an ER body or not is determined as follows. After introducing an expression vector into which DNA encoding the polypeptide is incorporated into a plant cell by a gene transfer technique known such as an electroporation, whether an ER body is formed or not in the plant cell can be determined by observation with a microscope or the like.

By there being an endomembrane system migration signal peptide at the N-terminus, protein synthesized in ribosome migrates into ER. In addition, by there being an ER retention signal peptide at the C-terminus, protein can stay in the ER. That is, in order to accumulate an expressed protein in an ER body, the condition that an endomembrane system migration signal peptide is present at the N-terminus of the protein, and an ER retention signal peptide is present at the C-terminus is required.

In the present invention, an endomembrane system migration signal peptide provided in the target protein is not particularly limited as long as a peptide has migration ability (hereinafter also referred to as ER migration ability) to endomembranes such as ER, and it can be appropriately selected from among signal peptides which are present at the N-terminus of secretory proteins and used. In addition, with respect to an endomembrane system migration signal peptide in the related art, as long as the ER migration ability is not impaired, a peptide may be one in which one or several amino acids are deleted, substituted, or added. Specific examples of an endomembrane system migration signal peptide include an endomembrane system migration signal peptide which is included in Pr1a protein of Tabacco mosaic virus (refer to Non-Patent Literature 10), an endomembrane system migration signal peptide (1st to 24th amino acid) of Pyk10 (seq id no. 2) of Arabidopsis thaliana, and an endomembrane system migration signal peptide (1st to 24th amino acid) of nai2 (seq id no. 1) of Arabidopsis thaliana and the like.

In the present invention, the ER retention signal peptide provided in the target protein is not particularly limited as long as the peptide has ER retention ability, and it can be appropriately selected from among signal peptides which are present at the C-terminus of the protein retained in the ER and used. Specific examples of the ER retention signal peptide include KDEL, HDEL, and the like in the amino acid one-letter code.

In the protein retained in the ER, at least a portion of an endomembrane system migration signal peptide at the N-terminus is cleaved by the enzyme of the ER. In the present invention, depending on the type of an endomembrane system migration signal peptide provided in a target protein, instead of the target protein, the protein in which an N-terminal region of the target protein is lacking may be accumulated in the ER body which is formed. Sites cleaved by enzyme in ER vary depending on the type of the target protein, in particular, the amino acid sequence of the polypeptide or the like which is ligated to the endomembrane system migration signal peptide. In many cases, only the endomembrane system migration signal peptide is lacking due to the cutting, but there is also a case in which a wider N-terminus region including an endomembrane system migration signal peptide is lacking, or only a portion of the endomembrane system migration signal peptide is lacking.

In a case where a desired protein to be accumulated in cells originally has an endomembrane system migration signal peptide and an ER retention signal peptide, by co-expressing a gene which encodes the protein with the ER body formation related gene, the protein or a protein in which N-terminus region of the protein is lacking can be accumulated in the ER body. On the other hand, in a case where a desired protein to be accumulated in a cell does not have an endomembrane system migration signal peptide and an ER retention signal peptide, by setting a protein, as a target protein, with an added endomembrane system migration signal peptide at the N-terminus, and an ER retention signal peptide at the C-terminus of the protein respectively, the target protein or a protein in which N-terminus region of the protein is lacking can be accumulated in an ER body.

In the present invention, a target protein may be a chimeric protein in which a protein with an added ER retention signal peptide in a desired protein to be accumulated in a cell is fused directly or via a suitable spacer at the C-terminus of the protein originally having an endomembrane system migration signal peptide at the N-terminus. For example, a protein in which another polypeptide is fused at the C-terminus of Pyk10 may be a target protein.

The method for co-expressing a protein having an ability to form an ER body in a plant cell and a target protein is not particularly limited, and may be performed by any method known in the related art. For example, by introducing an expression vector having a base sequence which encodes a protein having an ability to form an ER body and an expression vector having a base sequence which encodes a target protein in a plant cell, it is possible to prepare transformed cells in which a protein having an ability to form an ER body and a target protein are co-expressed. An expression vector having both a base sequence which encodes a protein having an ability to form an ER body and a base sequence which encodes a target protein may be introduced.

A plant cell in which an ER body formation related gene and a gene which encodes the target protein are co-expressed may be a cell in a plant individual, a cell collected from a plant individual, a cell treated with dedifferentiation treatment or the like, and a cultured cell. By introducing an ER body formation related gene and a gene which encodes a target protein in the callus obtained by dedifferentiation treatment, it is possible to obtain a plant individual having a cell in which the target protein in an ER body which is formed or a protein in which the N-terminus region of the target protein is lacking is accumulated.

In the present invention, the type of plant cells in which an ER body formation related gene and a gene which encodes a target protein are co-expressed is not particularly limited, however, a cell of a plant species in which an ER body is not formed in the wild-type is preferable. Among them, a monocotyledonous plant is preferable, and liliaceae plants or gramineous plants are more preferable. Examples of liliaceae plants include onions and the like. In addition, examples of gramineous plants include rice, mealie, sorghum, wheat, barley, rye, millet, Eriansasu, sugar cane, switch grass, miscanthus, napier grass and the like.

An expression vector having a base sequence which encodes a protein having an ability to form an ER body and a target protein can be prepared by incorporating the DNA having a base sequence which encodes these proteins in the expression vector using well-known gene recombination techniques. A commercially available expression vector manufacturing kit also may be used.

The expression vector is not particularly limited as long as it has a promoter sequence capable of being transcribed in a plant cell and a terminator sequence including a polyadenylation portion, and when introduced in a plant cell, the vector is capable of expressing a polypeptide which is encoded by polynucleotides incorporated. Any expression vectors commonly used for preparing a transformed plant cell and a transformed plant may be used. In a case of incorporating a base sequence which encodes a protein having an ability to form an ER body and a base sequence which encodes a target protein into one expression vector, a cassette for expression formed of DNA having a promoter sequence, DNA having a base sequence which encodes a protein having an ability to form an ER body, and DNA having a terminator sequence, and a cassette for expression formed of DNA having a promoter sequence, DNA having a base sequence which encodes a target protein, and DNA having a terminator sequence are needed such that both proteins are expressed in a cell independently.

Examples of an expression vector include MultiRound Gateway (refer to Non-Patent Literature 11) entry vector and binary vector such as pIG121, pIG121Hm and the like. Examples of a usable promoter include a promoter of the nopaline synthase gene, a promoter of cauliflower mosaic virus 35S, a promoter of mealie ubi1 and the like. Further, examples of a usable terminator include a terminator and the like of nopaline synthase gene. In addition, a specific promoter in tissues and organs may be used. Examples of a leaf-specific expression promoter include a promoter of rice rbcS and the like. By using such tissues or organ-specific promoters, it is possible to express the target protein only in a specific tissue or organ, not in the whole plant.

For example, in a case of preparing a transformed plant by introducing an expression vector having a base sequence which encodes a protein having an ability to form an ER body and an expression vector having a base sequence which encodes a target protein into edible plants, it is possible to express the target protein only in the non-edible parts of the transformed plant.

The expression vector is preferably an expression vector in which not only DNA having a base sequence which encodes a protein having an ability to form an ER body and a target protein but also a drug resistance gene are incorporated. This is because the selection of plants which are transformed by the expression vector and plants which are not transformed can be easily performed. Examples of the drug resistance gene include a kanamycin resistance gene, a hygromycin resistance gene, a bialaphos resistance gene and the like.

In the present invention, a method for producing a transformed plant using an expression vector is not particularly limited, however, this can be performed by a method generally used in a case of producing a transformed plant cell and a transformed plant. Examples of the method include an Agrobacterium method, a particle gun method, an electroporation method, a PEG (polyethylene glycol) method, or the like. Among them, the Agrobacterium method is preferably performed. A transformed plant cell and a transformed plant can be selected by an indicator of drug resistance or the like. Further, as a host, a plant cultured cell may be used, or plant organs and plant tissue may be used.

By using a well-known plant tissue culture method, it is possible to obtain a transformed plant from transformed plant cells, a callus or the like. For example, by culturing transformed plant cells using a hormone-free redifferentiation medium or the like, transplanting an obtained rooted seedling plant into soil and cultivating, it is possible to obtain a transformed plant.

For example, a rice transformed such that an ER body related gene and a gene which encodes a target protein are co-expressed can be prepared by transforming an expression vector having a base sequence which encodes a protein having an ability to form an ER body and an expression vector having a base sequence which encodes a target protein with a commonly used method such as a method of Nishimura et al. (refer to Non-Patent Literature 12) and the like. Specifically, for example, a callus obtained by culturing the mature seeds which are surface-sterilized after removing a hull is made to be infected by immersing in a solution of Agrobacterium transformed by an expression vector having a base sequence which encodes a protein having an ability to form an ER body and an expression vector having a base sequence which encodes a target protein. Additionally, then, the callus which is transformed using antibiotic or the like is selected. Thus, it is possible to obtain rice which is a transformed plant of the present invention.

The transformed plant of the present invention obtained in this manner may be cultivated, grown from a cutting, or crossed or the like in the same manner as an individual plant prior to transformation, whereby progeny individuals may be obtained. It is also possible to obtain a cloned individual plant by cloning techniques known in the art.

In the transformed plant of the present invention, the target protein in the ER body which is newly formed as a protein storage organ or a protein in which the N-terminus region of the target protein is lacking are accumulated. Therefore, it is possible to protect a target protein or the like, for example, against proteolytic enzymes or the like present in the vacuoles, and accumulate stably. At the same time, adverse effects on other subcellular organelles, and furthermore, on plant growth caused by a target protein or the like can be sufficiently decreased.

In the transformed plant of the present invention, the target protein accumulated in the ER body which is newly formed or a protein in which the N-terminus region of the target protein is lacking may be recovered. A method for recovering a target protein or the like from a transformed plant of the present invention is not particularly limited; however, it can be performed by a method appropriately selected from methods generally used in a case of extracting and purifying recombinant proteins from cells or biological tissues. Examples of the method include a method of Kawazu et al. (refer to Non-Patent Literature 13), and a method of Kimura et al. (refer to Non-Patent Literature 14) or the like.

In addition, as a target protein in a method for accumulating a protein in a plant cell of the present invention, by using a diastatic enzyme which hydrolyses plant cell wall-derived cellulose to sucrose, for example, hyperthermophilic glucanase such as Acidothermus cellulolyticus derived endoglucanase E1 gene catalytic domain (E1-cat) (refer to Non-Patent Literature 15) and Pyrococcus furiosus derived β-glucosidase CelB gene (refer to Non-Patent Literature 16), it is possible to produce a transformed plant which becomes a biomass material suitable for bio-ethanol production. In the transformed plants obtained, since a diastatic enzyme is accumulated in the ER body of the transformed plants, it is possible to cultivate the diastatic enzyme together with the plant which became the host during the transformation. Moreover, in a case where the transformed plant is a biomass material, by subjecting to pretreatment for bio-ethanol production, as a result that a diastatic enzyme accumulated from the ER body is released, cellulose of the transformed plants is easily decomposed.

### EXAMPLES

Although the present invention will be described in further detail by showing the following examples, the present invention is not limited to the following examples.

### Example 1

By temporarily co-expressing an nai2 gene and a gene which encodes a target protein in onion epidermal cell, the target protein was accumulated in an ER body which was newly formed.

<Preparation of Gene Expression Vector>

Corn ubi1 promoter and Agrobacterium nos terminator were ligated with only a Aequorea victoria green fluorescent protein (GFP) gene which is a reporter, or with a Aequorea victoria green fluorescent protein (GFP) gene fused with Acidothermus cellulolyticus derived endoglucanase E1 gene catalytic domain (E1-cat) which is a diastatic enzyme (refer to Non-Patent Literature 15) or Pyrococcus furiosus derived β-glucosidase CelB gene (refer to Non-Patent Literature 16), as a fused protein, by a PCR method, and thus a gene expression cassette was prepared.

Tobacco mosaic virus Pr1a protein signal peptide (refer to Non-Patent Literature 10) was added at the code region 5′ terminal, and 4 amino acid residues (HDEL) which is an ER retention signal peptide was added at the code region 3′ terminal of each gene.

Further, corn ubi1 promoter, GFP gene, and Agrobacterium nos terminator were ligated by a PCR method, and a gene expression cassette (GFP expression cassette) which did not include any of an endomembrane system migration signal peptide and an ER retention signal peptide was prepared. In addition, a gene expression cassette of a nai2 gene (nai2 expression cassette) which is an Arabidopsis thaliana ER body formation related gene was prepared in the same manner.

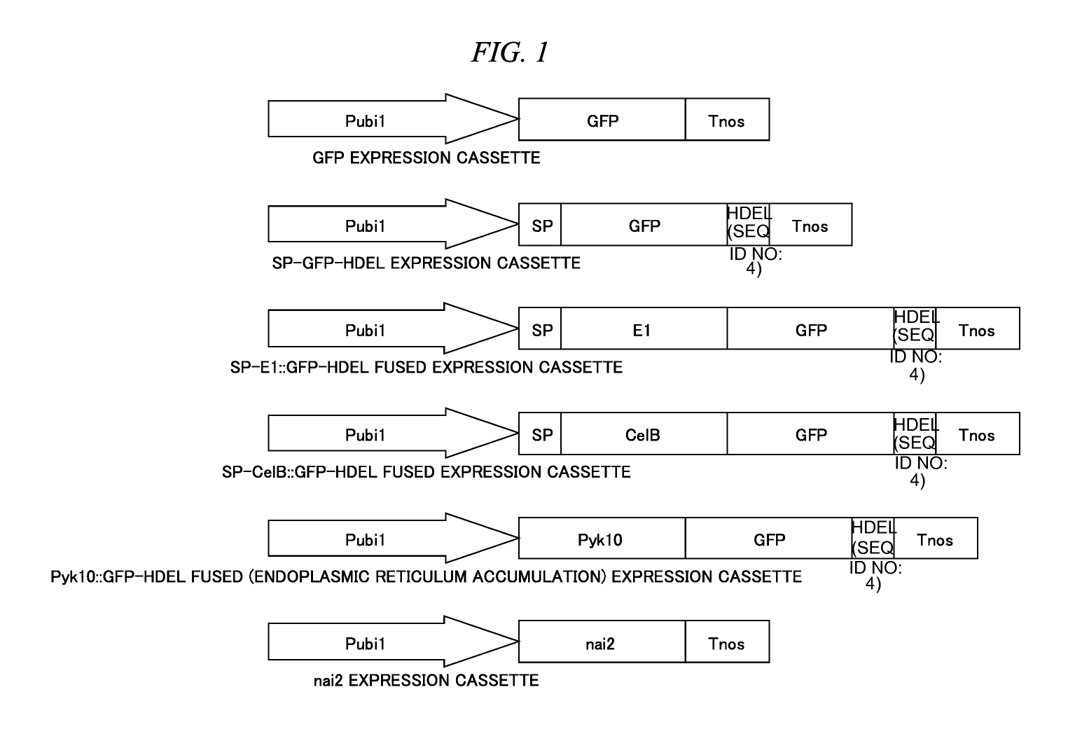

FIG. 1 is a diagram schematically showing each expression cassette prepared. In FIG. 1, “Pubi1” represents corn ubil1 promoter, “Tnos” represents Agrobacterium nos terminator, “HDEL” represents an ER retention signal peptide, “SP” represents Tobacco mosaic virus Pr1a protein signal peptide, “GFP” represents GFP, “E1” represents E1-cat, and “CelB” represents CelB, respectively.

By inserting the expression cassettes in MultiRound Gateway (refer to Non-Patent Literature 11) entry vector using In-Fusion Advantage PCR Cloning Kit (manufactured by Clontech Laboratories Inc), an expression vector was prepared.

<Gene Introduction by Particle Gun Method>

Using a gene recombination apparatus (particle gun) (Trade name: PDS-1000/He, manufactured by Bio-Rad Laboratories, Inc), a prepared expression vector was introduced into an onion bulb as described in the instruction manual.

<Observation of Cell into which an Expression Vector was Introduced>

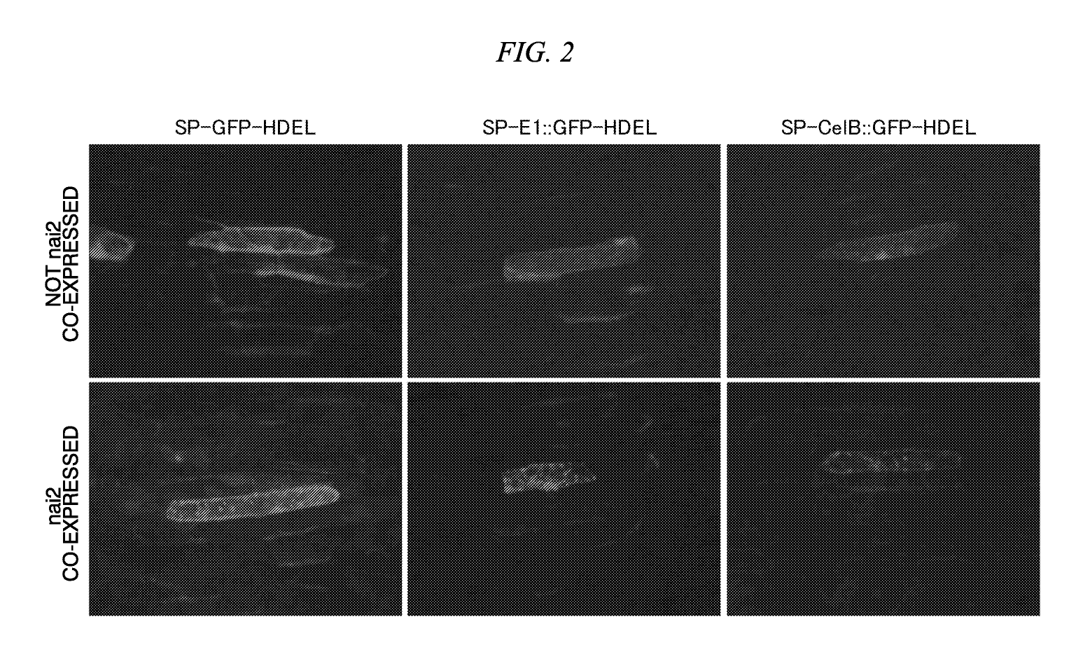

A cell into which an expression vector was introduced was observed using a fluorescence stereomicroscope and confocal laser scanning microscope. Specifically, by detecting fluorescence of GFP, localization of GFP or GFP fused protein expressed in the cell was observed. FIG. 2 is a fluorescence stereomicroscope image. In FIG. 2, the upper image (not nai2 co-expressed) is an image of a cell into which an expression vector including a nai2 expression cassette was not introduced at the same time, and the lower image (nai2 co-expressed) is an image of a cell into which an expression vector including a nai2 expression cassette was introduced at the same time.

As a result, in the cells into which an expression vector including the GFP expression cassette containing neither an endomembrane system migration signal peptide, nor an ER retention signal peptide was introduced, regardless of whether or not an expression vector including an nai2 expression cassette was introduced at the same time, the GFP was localized throughout the cytoplasm (not shown). On the other hand, in the cell into which an expression vector including SP-GFP-HDEL expression cassette having any of an endomembrane system migration signal peptide and an ER retention signal peptide, SP-E1::GFP-HDEL fused expression cassette, and SP-CelB::GFP-HDEL fused expression cassette was introduced, in a case where an expression vector including nai2 expression cassette was not introduced at the same time, each GFP or GFP fused protein was distributively localized through the entire ER. However in the cell into which an expression vector including a nai2 expression cassette was introduced at the same time, each GFP or GFP fused protein was localized in a subcellular organelle showing strong fluorescence intensity with an elliptical shape. The subcellular organelle is an ER body, by co-expressing nai2 and protein having an endomembrane system migration signal peptide and an ER retention signal peptide, an ER body is newly formed, and a protein having an endomembrane system migration signal peptide and an ER retention signal peptide was accumulated in an ER body.

### Example 2

A protein fused with another polypeptide at the C-terminus of Pyk10 protein was set as a target protein, and by the method for accumulating a protein in a plant cell of the present invention, a target protein was accumulated by forming an ER body, in onion epidermal cells.

<Preparation of Gene Expression Vector>

A fusion protein to which the GFP is ligated at the C-terminus of Pyk10, corn ubi1 promoter, and Agrobacterium nos terminator were ligated by a PCR method, thereby preparing a gene expression cassette. In the code region 3′ terminal of the gene, 4 amino acid residues (HDEL) which is an ER retention signal peptide was added. Moreover, as the above-described, an endomembrane system migration signal peptide was originally provided at the N-terminus of Pyk10. FIG. 1 is a schematic diagram of an expression cassette prepared (Pyk10::GFP-HDEL fused expression cassette).

By inserting Pyk10::GFP-HDEL fused expression cassettes in a MultiRound Gateway (refer to Non-Patent Literature 11) entry vector using In-Fusion Advantage PCR Cloning Kit (manufactured by Clontech Laboratories Inc), an expression vector was prepared.

<Observation of Cells into which a Gene and an Expression Vector are Introduced by a Particle Gun Method>

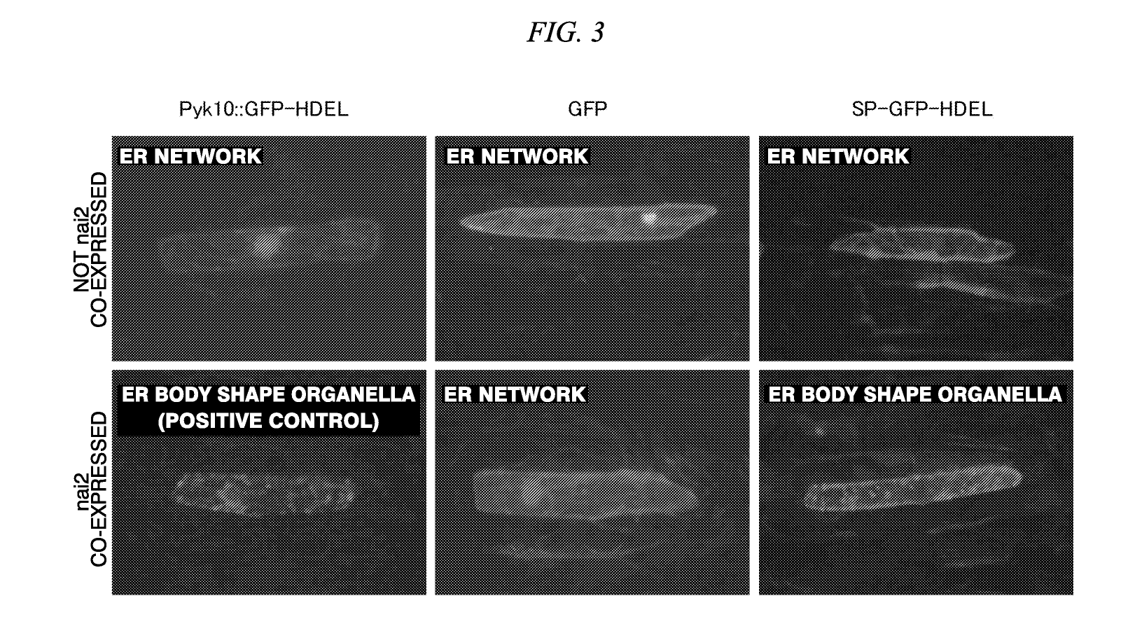

In the same manner as Example 1, an expression vector including Pyk10::GFP-HDEL fused expression cassette, an expression vector including SP-GFP-HDSL expression cassette used in Example 1, or an expression vector including an GFP expression cassette including neither an endomembrane system migration signal peptide nor an ER retention signal peptide, and an expression vector including a nai2 expression cassette were introduced into an onion bulb respectively. Further, in the same manner as Example 1, a cell into which an expression vector was introduced was observed by a fluorescence stereomicroscope and a confocal laser scanning microscope. FIG. 3 is fluorescence stereomicroscope images. In FIG. 3, an upper image (non nai2 co-expression) is an image of a cell into which an expression vector including a nai2 expression cassette was not introduced at the same time, and a lower image (nai2 co-expression) is an image of a cell in which an expression vector including a nai2 expression cassette was introduced at the same time.

As a result, in a case where an expression vector including nai2 expression cassette was not introduced at the same time, GFP fluorescence was observed through the entire ER in both a cell into which an expression vector including SP-GFP-HDSL expression cassette was introduced and a cell into which an expression vector including a Pyk10::GFP-HDEL fused expression cassette was introduced. In contrast, in a case where an expression vector including SP-GFP-HDEL expression cassette and an expression vector including nai2 expression cassette were introduced at the same time, the formation of an ER body was observed in about half the number of cells (that is, cells into which a gene was introduced) in which GFP fluorescence was observed. On the other hand, in a case where an expression vector including a Pyk10::GFP-HDEL fused expression cassette and an expression vector including a nai2 expression cassette were introduced at the same time, the formation of an ER body was observed in almost all cells in which GFP fluorescence was observed. In cells into which an expression vector including a GFP expression cassette containing neither an endomembrane system migration signal peptide nor an ER retention signal peptide was introduced, regardless of whether or not an expression vector including a nai2 expression cassette was introduced at the same time in the same manner as Example 1, the GFP was localized throughout the cytoplasm. From a result of the above, by fusing a desired protein with Pyk10 and expressing, and expressing nai2 at the same time, it is possible to form an ER body in a cell with high frequency.

### INDUSTRIAL APPLICABILITY

The present invention can provide a method which stably accumulates a desired protein in a plant cell and a plant, and a transformed plant in which a protein is accumulated. By a method for accumulating a protein into a plant cell of the present invention, since the effect on a plant cell and a plant individual resulting from an overexpression can be sufficiently decreased while the desired protein can be accumulated in a plant cell, the method or transformed plants of the present invention obtained by the method can be used, for example, in the field of mass production of the protein or trait modifications of plants and the like.

1. A method for accumulating a protein in a plant cell,
wherein by co-expressing a gene which encodes a protein having an ability to form an endoplasmic reticulum (ER) body, and a gene which encodes a target protein having an endomembrane system migration signal peptide at an N-terminus and having an ER retention signal peptide at a C-terminus in the plant cell, the target protein or a protein in which the N-terminus domain of the target protein is lacking is accumulated in the ER body formed in the plant cell.

2. The method for accumulating a protein in the plant cell according to claim 1,
wherein a protein having the ability to form an ER body is a polypeptide selected from any one of (a) to (d) below:
(a) a polypeptide formed of an amino acid sequence represented by SEQ ID NO: 1,
(b) a polypeptide formed of an amino acid sequence in which one or several amino acids are lacking, substituted or added, and having the ability to form an ER body, in the amino acid sequence represented by SEQ ID NO: 1,
(c) a polypeptide having homology of 80% or more with the amino acid sequence represented by SEQ ID NO: 1 and having the ability to form an ER body, and
(d) a polypeptide having homology of 80% or more with amino acid sequence formed of the 473rd to the 772nd amino acid in the amino acid sequence represented by SEQ ID NO: 1 and having the ability to form an ER body.

3. The method for accumulating a protein in the plant cell according to claim 1, wherein the target protein is a protein fused with another polypeptide at the C-terminus of Pyk10 protein.

4. The method for accumulating a protein in a plant cell according to claim 1, wherein the plant cell is a cell in plant individual.

5. A transformed plant,
wherein an expression vector having a base sequence which encodes a protein having an ability to form an endoplasmic reticulum (ER) body and an expression vector having a base sequence which encodes a target protein are introduced, or an expression vector having both a base sequence which encodes a protein having an ability to form an ER body and a base sequence which encodes a target protein is introduced,
the target protein has an endomembrane system migration signal peptide at the N-terminus, and has an ER retention signal peptide at the C-terminus, and
in at least one cell in the plant, a target protein or a protein in which an N-terminus region of the target protein is lacking is accumulated in an ER body formed in the cell.

6. The transformed plant according to claim 5,
wherein the protein having an ability to form an ER body is a polypeptide selected from any one of (a) to (d) below:
(a) a polypeptide formed of the amino acid sequence represented by SEQ ID NO: 1,
(b) a polypeptide formed by an amino acid sequence in which one or several amino acids are lacking, substituted or added, and having the ability to form an ER body, in the amino acid sequence represented by SEQ ID NO: 1,
(c) a polypeptide having homology of 80% or more with the amino acid sequence represented by SEQ ID NO: 1 and having the ability to form an ER body, and
(d) a polypeptide having homology of 80% or more with the amino acid sequence formed of the 473rd to the 772nd amino acid in the amino acid sequence represented by SEQ ID NO: 1, and having the ability to form an ER body.

7. The transformed plant according to claim 5, wherein the target protein is a protein fused with another polypeptide at the C-terminus of Pyk10 protein.

8. The transformed plant according to claim 5, which is a monocot.

9. The transformed plant according to claim 5, which is one of a liliaceae plant and a gramineous plant.

10. The transformed plant according to claim 5, which is a rice plant.

11. A plant which is a next generation individual or a cloned individual of the transformed plant according to claim 5.

12. The method for accumulating a protein in the plant cell according to claim 2, wherein the target protein is a protein fused with another polypeptide at the C-terminus of Pyk10 protein.

13. The method for accumulating a protein in a plant cell according to claim 2, wherein the plant cell is a cell in plant individual.

14. The method for accumulating a protein in plant cell according to claim 3, wherein the plant cell is a cell in plant individual.

15. The transformed plant according to claim 6, wherein the target protein is a protein fused with another polypeptide at the C-terminus of Pyk10 protein.

16. The transformed plant according to claim 6, which is a monocot.

17. The transformed plant according to claim 6, which is one of a liliaceae plant and a gramineous plant.

18. The transformed plant according to claim 6, which is a rice plant.

19. A plant which is a next generation individual or a cloned individual of the transformed plant according to claim 6.

20. A plant which is a next generation individual or a cloned individual of the transformed plant according to claim 7.
